(19) United States
(12) Patent Application Publication
(10) Pub. No.: US 2009/0209948 A1
(43) Pub. Date: Aug. 20, 2009

(54) Laser Handpiece for Treatment of the Human Body and a Controlling Laser Radiation Emission

(76) Inventor: Marcello Rinaldo Baldacchini, Brescia (IT)

Correspondence Address: SHOEMAKER AND MATTARE, LTD, 10 POST OFFICE ROAD - SUITE 100, SILVER SPRING, MD, 20910, US

(21) Appl. No.: 12/301,091

(22) PCT Filed: Apr. 24, 2007
(86) PCT No.: PCT/IT2007/000301

§ 371 Date: Nov. 17, 2008

(30) Foreign Application Priority Data
May 17, 2006 (IT) BS2006A000106

(51) Int. Cl. A61B 18/20; H01S 5/42

(52) U.S. Cl. 606/13; 372/50.12

(57) ABSTRACT

The object of the present invention is a laser handpiece (1) for the treatment of deep regions of the human body. There is provided a plurality of emitters (30) arranged so that the emission cones remain disconnected at least up to the treatment depth. Moreover, the object of the present invention is a method for controlling the emission of laser radiations, which comprises the step of determining the emission parameters (switch on frequency and/or switch on time and/or emission intensity modulation) based on the patient's biological parameters (heart rate and/or breathing rate and/or blood pressure).

The object of the present invention is a laser light emitting handpiece for the treatment of a part of the human body, especially for medical purposes. The object of the present invention further is a method for controlling laser radiation emission from a handpiece for the treatment of the human body.

Several laser handpieces currently exist, used for the treatment of the human body, especially for treating pathologies like muscle strains, tendinitis, discopathy and the like.

These pathologies affect deep regions of the human body, that is, regions far away from the epithelium surface; for this reason, the radiations emitted by usual LEDs are not suitable for carrying out such treatments as they are incoherent. LED handpieces can therefore be used for the treatment of surface regions of the human body or of the skin surface, for medical or aesthetic purposes.

On the other hand, deep regions may be treated with laser emitters that, as known, emit a coherent radiation, capable of penetrating deep into the human body and impinging the regions concerned.

However, laser handpieces used nowadays exhibit some disadvantages. Above all, they are not capable of impinging deep regions of the human body with sufficient energy for allowing them suitable treatment.

The object of the present invention is to obtain a laser light emitting handpiece which should overcome the disadvantages mentioned above with reference to the prior art.

The features and advantages of the laser handpiece according to the present invention will appear more clearly from the following description, made by way of an indicative and non-limiting example with reference to the following figures, wherein:

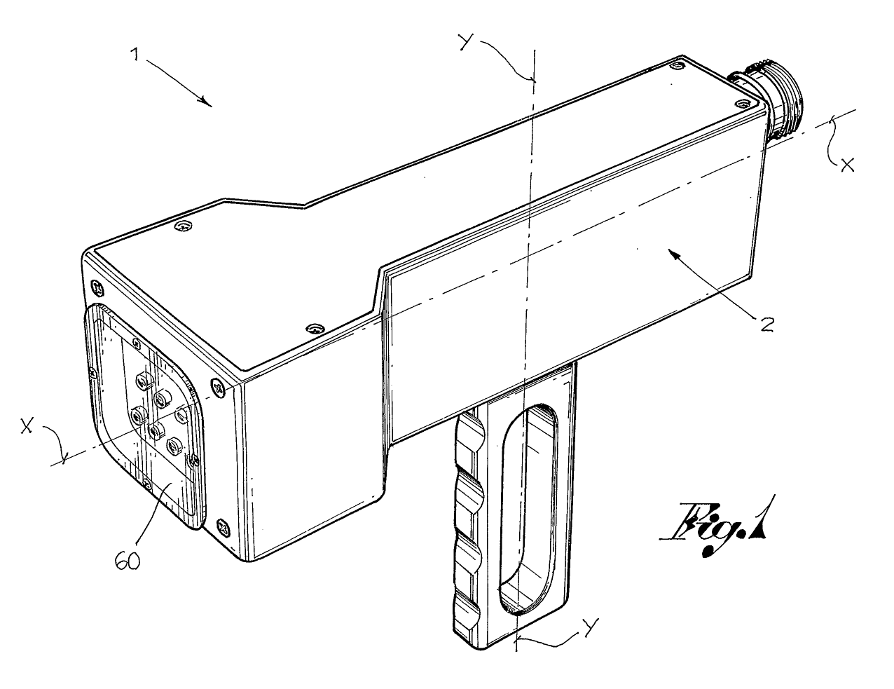

FIG. 1 shows a perspective view of the handpiece according to an embodiment variation of the present invention;

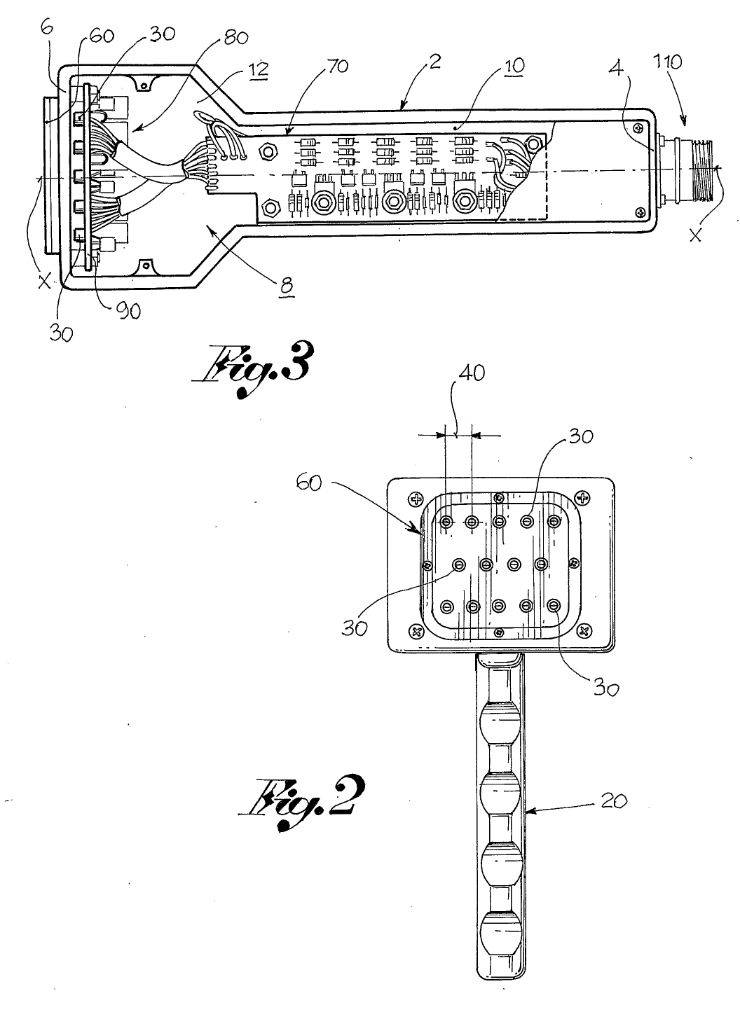

FIG. 2 shows a front view of the handpiece of FIG. 1;

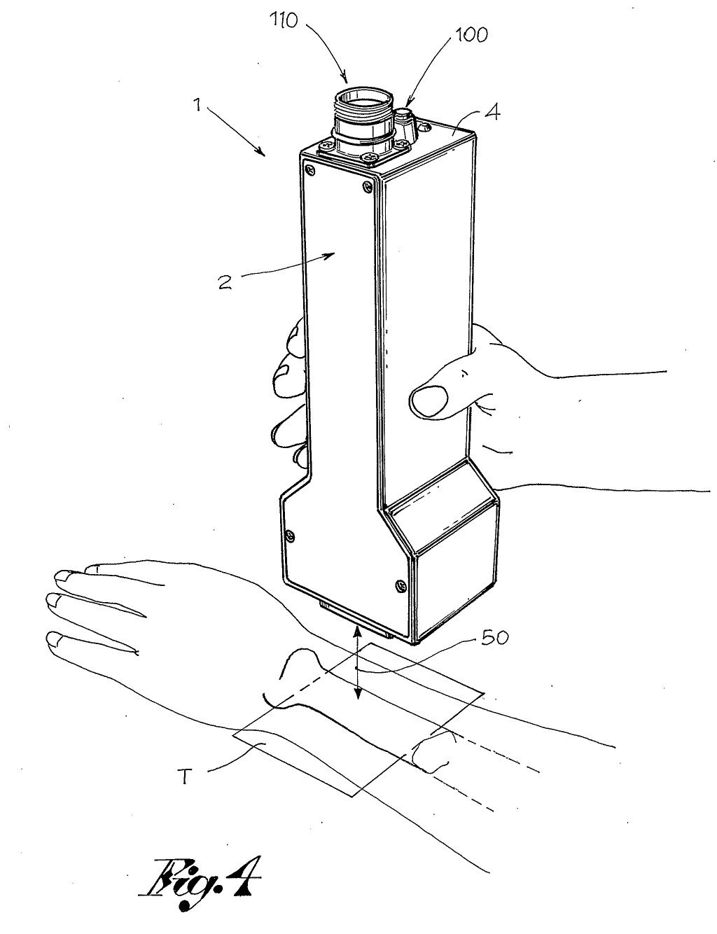

FIG. 3 shows a plan view of the interior of the handpiece of FIG. 1; and

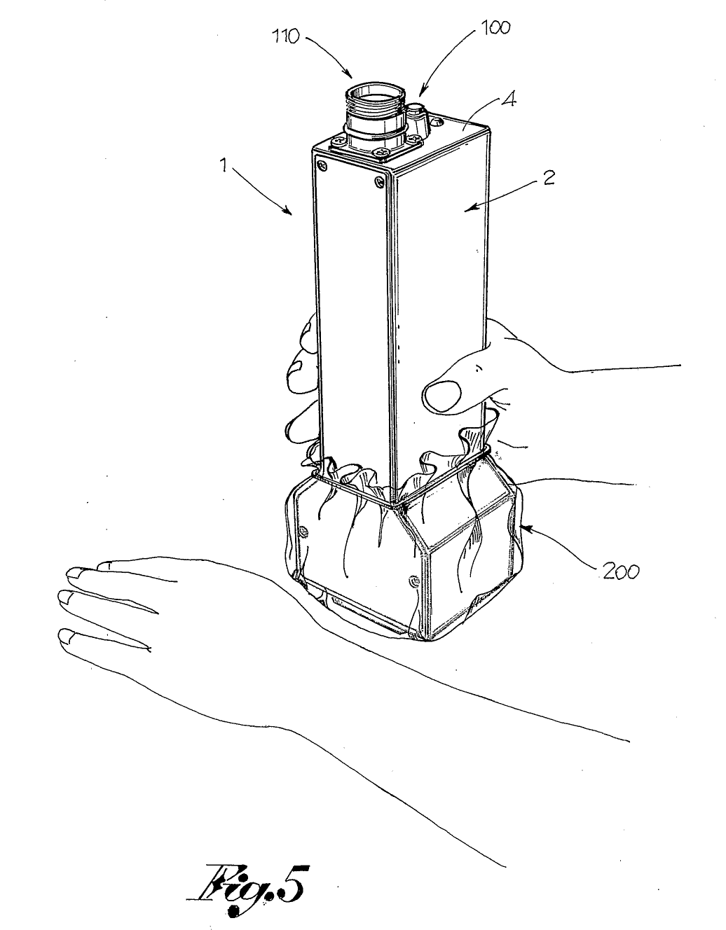

FIG. 4 shows a perspective view of a further embodiment variation of the handpiece according to the invention.

With reference to the annexed figures, reference numeral 1 globally denotes a laser handpiece suitable for being moved on the surface of a human body zone for treating body regions located at a treatment depth from the surface of the human body zone.

Handpiece 1 comprises an enclosure 2 suitable for being grasped, for example manually by an operator, and moved on said human body zone.

Enclosure 2 is hollow and mainly extends along an enclosure axis X-X, between a back wall 4 and a front wall 6.

Enclosure 2 internally exhibits a cavity 8 wherein there are an elongated back space 10 and a front space 12, larger than the back space 10, but shorter than it. The enclosure is preferably made of aluminium.

Handpiece 1 further comprises a handgrip 20, removably associated to enclosure 2 for giving a “gun” shape. In other words, the handgrip mainly extends along a gripping axis Y-Y, incident to the enclosure axis X-X, for example perpendicular thereto.

Handpiece 1 further comprises laser emitting means suitable for emitting laser radiations having a predetermined wavelength.

Preferably, said wavelength is comprised between 905 nanometres and 935 nanometres, that is, substantially equal to the characteristic silicon wavelength.

The laser emitting means are mounted on enclosure 2, for emitting the laser radiations towards said human body zone.

The laser emitting means comprise a plurality of emitters 30. Each emitter is suitable for emitting said laser radiations according to an emission cone.

Said emission cone has an elliptical section. In other words, placing a geometrical plane in front of the emitters, perpendicular to the direction of emission of said emitters, the mark left by the emission cone of emitters 30 on said geometrical plane is elliptical.

In yet other words, once a vertical plane has been defined as the plane perpendicular to the plane on which the emitter lays and containing the emission axis and a horizontal plane has been defined as the plane perpendicular to the vertical plane and containing the emission axis, the radiation emitted by each emitter 30 exhibits a vertical divergence angle relative to the emission axis on the vertical plane and a horizontal divergence angle relative to the emission axis on the horizontal plane.

Preferably, emitter 30 is of the “single chip” type, based on gallium arsenide, and they are suitable for emitting with a vertical divergence angle of twenty hexagesimal degrees and a horizontal divergence angle of ten hexagesimal degrees.

Emitters 30 are arranged at a relative distance 40 from each other such as to keep said emission cones disconnected from each other at least up to a treatment plane T arranged at a distance 50 from said emitters 30 equal to said treatment depth.

Preferably, said relative distance 40 between emitters 30 is such that on the treatment plane T, the marks of the emission cones are tangent to each other.

Advantageously, such feature allows keeping the radiation emitted by each emitter coherent at least up to the treatment plane, substantially coincident with the body region to be treated. At a higher depth than that of treatment, in fact, the emission cones tend to intersect, so the radiation of each emitter, not in phase with the radiation of the other emitters, combines with such radiation emitted by the other emitters, generating an incoherent radiation.

Advantageously, the action of coherent radiations on the body region to be treated is much more effective than the action promoted by incoherent radiations.

According to a preferred embodiment, emitters 30 are arranged according to parallel rows, for example as beehives, that is, so that the emitters of a row are arranged in median position relative to the emitters of the row immediately subsequent or preceding.

Advantageously, on the treatment plane, such arrangement of the emitters allows obtaining a high index of filling, intended as ratio between the extension of the surface impinged by the radiations coming from all the emitters as compared to the extension of the surface not impinged.

By relative distance 40 it is meant the distance between emitters 30 arranged on the same row, whereas by “distance between rows” it is meant the distance between one row and the subsequent or preceding one.

Preferably, the relative distance 40 is different from the distance between the rows. For example, the relative distance 40 between the emitters of one row is equal to 8.5 millimetres whereas the distance between the rows is equal to 16 millimetres. Such distances are designed on the basis of the vertical and horizontal divergence angles that characterise the emitters and on the basis of the treatment depth permitted by said emitters.

According to a preferred embodiment, said laser emitting means comprise fourteen emitters 30, and in particular five arranged on the first row, four arranged on the second row and five arranged on the third row.

According to a preferred embodiment, said handpiece further comprises a support wall 60, arranged in front of emitters 30, at a predetermined distance therefrom. Said support wall 60 is transparent to the radiations emitted by emitters 30, whereas preferably, it is opaque to ordinary light.

When handpiece 1 is put on the human body zone, the support plate 60 rests on the skin of such zone, thus fixing also the minimum distance between the skin and emitters 30.

According to a preferred embodiment, handpiece 1 comprises control means suitable for controlling said laser emitting means, preferably seated in enclosure 2 of handpiece 1.

In particular, said control means comprise pre-piloting means 70 suitable for controlling the switch on time, the switch on intensity and the switch on frequency of emitters 30. Preferably, said piloting means are seated in the back space 10 of enclosure 2.

Moreover, the control means comprise piloting means 80, suitable for powering said emitters 30 for creating the laser emission. Preferably, said piloting means are seated in the front space 12 of enclosure 2.

Moreover, handpiece 1 comprises a support plate 90, operatively connected to said control means, on which emitters 30 are fixed. The support plate 90 is seated in the front space 12 of enclosure 2, in front of the front wall 6 thereof, whereas emitters 30 are facing outwards through a window obtained in said front wall 6 of the enclosure.

According to a preferred embodiment, the piloting means 80 are seated on said support plate 90.

Advantageously, such construction allows avoiding heat dissipations due to the passage of high currents in electrical wires connecting the piloting means 80 and emitters 30, as it happens in known embodiments.

According to a preferred embodiment, handpiece 1 comprises an on-off switch 100, located for example on the back wall 6 of enclosure 2. Switch 100 is suitable for being pressed for controlling the emission of laser radiations; preferably, when switch 100 is released, the emission of laser radiations continues, until it is pressed again.

According to an embodiment variation, said switch is suitable for being pressed for controlling the emission of laser radiations and, when released, the emission of laser radiations is interrupted.

Advantageously, the position of switch 100 allows conveniently using handpiece 1 both in the “gun” configuration, that is, with handgrip, and without handgrip, grasping the entire enclosure in the palm and four fingers, at the back space 10 thereof, and pressing the switch with the thumb.

According to a further preferred embodiment, handpiece 1 comprises a connection socket 110, located on the back wall 10 of enclosure 2, suitable for connecting handpiece 1 to a control console (not shown).

The control console comprises handpiece management means, for example for determining the working parameters of the handpiece, that is, the switch on time, the switch on frequency and the switch on intensity modulation.

According to a preferred embodiment, said console comprises working parameter input means, said input means being operatively connected to said means for managing handpiece 1.

In particular, said parameter input means are suitable for transmitting patient's biological parameters to said management means. In particular, said input means are suitable for transmitting the value of the heart rate and/or of the breathing rate and/or of the blood pressure to said management means.

In an embodiment variation, said input means are manual and comprise, for example, a keyboard for inputting numerical values.

In a further embodiment variation, said input means are automatic and suitable for continuously acquiring the values of said biological parameters and transmitting them to said management means.

For example, the automatic input means comprise a heart rate meter, for acquiring the heart rate, and/or a spirometer, for acquiring the breathing rate, and/or a blood pressure meter for acquiring the blood pressure.

In the standard use of handpiece 1, it is grasped by the operator. For example, if the patient is sitting and the treatment must be carried out on the back, it is particularly convenient to associate the handgrip to enclosure 2. The handpiece is repeatedly passed on the zone concerned, pressing the on-off switch 100 for emitting the laser radiations.

According to a further aspect of the present invention, the use of handpiece 1 envisages the execution of a method for controlling the emission of laser radiations.

The control method comprises the step of acquiring the patient's biological parameters.

Said biological parameters, that is, the heart rate and/or the breathing rate and/or the blood pressure may be measured by the operator or suggested by the patient, as they are known thereby.

Said biological parameters are manually entered by said input means and transmitted to said management means.

In a further embodiment variation of the control method, said biological parameters are continuously acquired and transmitted to the management means.

The control method further comprises the step of determining the emission parameters of laser radiations (that is, the switch on frequency, the switch on time and the emission intensity modulation) bases on said biological parameters.

In particular, the switch on frequency and/or the switch on time are determined on the basis of the heart rate and/or of the breathing rate, whereas the emission intensity modulation is determined on the basis of the blood pressure, for example proportional or inversely proportional thereto.

For example, for treatments of the human body that require toning of the cells of the region to be treated, it is particularly advantageous if the switch on frequency and the switch on time are determined based on the heart rate or the cardiogram pattern, which regulate the arterial system.

For treatments of the human body that require detoxination of the cells, it is particularly advantageous if the switch on frequency and the switch on time are determined based on the breathing rate, which regulates the venous system.

For treatments of the human body for which a benefit is obtained by an action also mechanical on the cells of the concerned region, it is particularly advantageous if the emission intensity modulation is determined based on the patient's blood pressure.

Innovatively, the handpiece according to the present invention and the method for controlling the emission of laser radiations usable therewith allow treating even deep regions of the human body with sufficient energy and with such methods as to achieve important results.

According to a usage variation, handpiece 1 is used in combination with a bag 200, completely transparent to laser radiations, arranged with a portion thereof between wall 60 of handpiece 1 and the patient's skin surface. The bag can be replaced.

Advantageously, said usage variation allows maintaining excellent hygienic conditions, since the bag is replaced before a treatment on a new patient is carried out.

For example, bag 200 is held on handpiece tight on the union between the portion of enclosure 2 relating to the front space 12 and the portion of enclosure 2 relating to the back space 10.

It is clear that a man skilled in the art can make changes and variations to the handpiece and to the control method described above, all falling within the scope of protection defined in the following claims.

1-38. (canceled)

39. Handpiece (1) suitable for being moved on the surface of a human body zone for treating body regions located at a treatment depth from the surface of the human body zone, comprising:
an enclosure (2) suitable for being grasped and moved on said human body zone;
laser emitting means suitable for emitting laser radiations having a predetermined wavelength, said emitting means being mounted on said enclosure (2) for emitting said radiations towards said human body zone;
wherein said laser emitting means comprise a plurality of emitters (30), each emitter being suitable for emitting said radiations according to an emission cone and said emitters being arranged at a relative distance (40) from each other
wherein said relative distance (40) is such as to keep said emission cones disconnected from each other at least up to a geometrical plane (T) arranged at a distance from said emitters equal to said treatment depth, characterised in that said wavelength is comprised in a range between 905 nanometres and 935 nanometres.

40. Handpiece according to claim 39, wherein said enclosure (2) is made of aluminium.

41. Handpiece according to claim 39, comprising control means suitable for controlling said laser emitting means.

42. Handpiece according to claim 39, wherein said control means are seated in said enclosure.

43. Handpiece according to claim 41, wherein said control means comprise pre-piloting means (70) suitable for controlling the switch on time, the switch on intensity and the switch on frequency of said emitters of the laser emitting means.

44. Handpiece according to claim 41, wherein said control means comprise piloting means (80) suitable for powering said emitters for creating said laser emission.

45. Handpiece according to claim 41, wherein said emitters are seated on a support plate (90), said support plate being seated in said enclosure, said emitters facing outwards for emitting said radiations towards said zone, said piloting means seating on said support plate.

46. Handpiece according to claim 43, wherein said pre-piloting means (70) and piloting means (80) are electrically connected to and spatially separated from each other.

47. Handpiece according to claim 39, wherein the emission cone has an elliptical section.

48. Handpiece according to claim 39, wherein said relative distance between the emitters is such that on said geometrical plane, the sections of said emission cones are tangent to each other.

49. Handpiece according to claim 39, wherein said emitters are arranged according to parallel rows.

50. Handpiece according to claim 39, wherein the emitters are arranged according as beehive.

51. Handpiece according to claim 49, wherein the emitters of a subsequent row are arranged in median position relative to the emitters of a preceding row.

52. Handpiece according to claim 49, wherein said relative distance is the distance between the emitters arranged along a row, said rows being spaced from each other by a distance between rows, said relative distance being different from said distance between rows.

53. Handpiece according to claim 49, wherein said emitters are arranged on three rows, of which the first row contains five emitters, the second row contains four emitters and the third row contains five emitters.

54. Handpiece according to claim 39, wherein said emitters are of the “single chip” type.

55. Handpiece according to claim 39, wherein said laser emitters are based on gallium arsenide.

56. Handpiece according to claim 39, comprising a handgrip (20) removably associable to said enclosure for making a “gun” handpiece.

57. Handpiece according to claim 39, comprising an on-off switch (100), said switch being arranged on the back of the enclosure, in a position opposite that occupied by said emitters.

58. Handpiece according to claim 57, comprising a connection socket (110) suitable for connecting said handpiece to a control console, said connection socket being arranged at the back of the enclosure.

59. Handpiece according to claim 39, comprising a support plate (60) transparent to laser radiations, arranged in front of said emitters for making a support plane of the handpiece on the human body zone.

60. Unit for the treatment of the human body comprising a control console and a handpiece made according to claim 39.

61. Unit according to claim 60, comprising input means suitable for inputting working parameters for controlling the emission of said radiations.

62. Unit according to claim 61, wherein said input means are seated on said console.

63. Assembly comprising a bag (200) transparent to laser radiations and a handpiece (1) made according to claim 39.

64. Assembly according to claim 63, wherein said bag is coupled to said handpiece so as to arrange with at least one portion thereof in front of said emitters.

65. Method for determining the relative distance (40) between emitters and the relative distance between parallel rows of emitters of a handpiece according to claim 39, said emitters having an emission cone with an elliptical section, comprising the steps of:
determining the distance between the emitters and the body region to be treated, located at a treatment depth from the surface of the human body zone on a treatment plane T;
determining the relative distance (40) between the emitters and the relative distance between parallel rows of emitters so that the marks of the emission cones on the treatment plane T are tangent to each other.

66. Method for controlling the emission of laser radiations for the treatment of a region of the human body of a patient, wherein said method comprises the steps of:
acquiring the patient's biological parameters;
inputting said biological parameters into a laser radiation emission unit for the treatment of said human body region;
determining the emission parameters of said laser radiations on the basis of said biological parameters;
emitting said laser radiations.

67. Method according to claim 66, wherein said acquisition step comprises the step of acquiring the patient's heart rate.

68. Method according to claim 67, wherein said determination step comprises the step of fixing the switch on time and the switch on frequency of said laser emitting means based on said heart rate.

69. Method according to claim 67, wherein said acquisition step comprises the step of continuously acquiring the patient's heart rate.

70. Method according to claim 66, wherein said acquisition step comprises the step of acquiring the patient's breathing rate.

71. Method according to claim 66, wherein said determination step comprises the step of fixing the switch on time and the switch on frequency of said laser emitting means based on said breathing rate.

72. Method according to claim 66, wherein said acquisition step comprises the step of continuously acquiring said patient's breathing rate.

73. Method according to claim 66, wherein said acquisition step comprises the step of acquiring the patient's blood pressure.

74. Method according to claim 73, wherein said determination step comprises the step of fixing the emission intensity of said laser emitting means based on said blood pressure.

75. Method according to claim 74, wherein said emission intensity is determined proportional to the patient's blood pressure.

76. Method according to claim 74, wherein said emission intensity is determined inversely proportional to the patient's blood pressure.

77. Method according to claim 73, wherein said acquisition step comprises the step of continuously acquiring the patient's blood pressure.
